(19) United States
(12) Patent Application Publication
(10) Pub. No.: US 2026/0139388 A1
(43) Pub. Date: May 21, 2026

(54) Dual Pressure Cryogenic Separation and Purification of Ethylene from Carbon Monoxide Electrolyzer Product

(71) Applicant: Dioxycle, Saint Ouen sur Seine (FR)

(72) Inventors: Jonathan Maistrello, La Garenne Colombes (FR); Paul S. Wallace, Paris (FR)

(21) Appl. No.: 19/370,694

(22) Filed: Oct. 27, 2025

Related U.S. Application Data
(60) Provisional application No. 63/723,587, filed on Nov. 21, 2024.

(51) Int. Cl. C25B 3/03; C07C 11/04; C25B 3/20

(52) U.S. Cl. CPC C25B 3/03; C07C 11/04; C25B 3/20

(57) ABSTRACT

This disclosure relates to systems and methods for separating the output stream of a carbon monoxide electrolyzer. A disclosed system includes a carbon monoxide electrolyzer which uses a dual-pressure cryogenic separation system to separate the output gas stream into three portions including a hydrogen-rich vapor stream, a CO-rich stream, and a purified ethylene stream. The cryogenic separation system includes a refrigeration system, a high-pressure gas/liquid separator, an expansion valve, and a low-pressure stripping column.

### CROSS REFERENCE TO RELATED APPLICATIONS

This application claims the benefit of U.S. Provisional Patent Application No. 63/723,587 as filed on Nov. 21, 2024, which is incorporated by reference herein in its entirety for all purposes.

### BACKGROUND

There is an urgent need to develop technologies for sustainable production of useful fuels and chemicals that does not rely on fossil resource extraction and processing for their production. Accordingly, technologies that both generate useful fuels and chemicals from alternative sources while using processes that reduce CO₂ emissions into the atmosphere are critically important. These technologies not only reduce net emissions but also create economic value by offsetting the costs of oxocarbon capture and conversion through the production of valuable chemicals.

One economically useful oxocarbon is carbon monoxide (CO), which can be valorized directly in an electrolyzer into olefins, alcohols, and carboxylates, but can also be used in Fischer-Tropsch synthesis and other methods to create a variety of other useful industrial chemicals. CO electrolyzers can operate using low-carbon footprint electricity by using renewable electricity and often operate with sustainable oxidation substrates such as water. Products of CO electrolyzers such as ethylene can be valuable, but there are remaining challenges in product separation. Purification of ethylene and other byproducts produced in an electrolyzer varies substantially from methods established for ethylene sourced from fossil fuel processing. CO₂ electrolyzers that directly produce ethylene also require a similar purification method.

### SUMMARY

This disclosure relates to systems and methods for producing and separating ethylene from an oxocarbon electrolyzer product stream. Carbon monoxide electrolyzers can be used to convert CO with low-carbon-footprint electricity into ethylene and hydrogen, along with unreacted carbon monoxide. The resulting product gas stream typically comprises ethylene, carbon monoxide, hydrogen, and, depending on the source gas, may also include other species such as nitrogen, methane, or carbon dioxide. A central challenge is the development of a dedicated separation process capable of producing high-purity ethylene suitable for downstream use while achieving high recovery rates and efficient recycling of CO and hydrogen. Carbon dioxide electrolyzers can be used to convert CO₂ into ethylene and hydrogen, along with a CO byproduct and unreacted CO₂. A similar separation process to produce high-purity ethylene from the output stream of CO₂ electrolysis can also recycle or otherwise process CO₂ before sending the output stream for further separation.

In traditional olefin production processes that produce ethylene (e.g., steam crackers, catalytic crackers, or methanol-to-olefin reactors), multiple cryogenic distillation columns are used to separate ethylene from other hydrocarbon and gas species. However, the product gas composition of oxocarbon electrolyzers is fundamentally different and contains significant concentrations of CO and hydrogen along with little or no C3 or higher hydrocarbons unless the electrolyzer catalyst is specifically chosen for that purpose. This makes conventional cracking-based purification schemes inefficient or inapplicable. Moreover, conventional separation schemes often direct incompletely separated CO, CO₂, and hydrogen into fuel or flare streams, which is incompatible with recycling strategies that maximize conversion efficiency in an electrolyzer. High-pressure cryogenic separators used therein can recover most of the lower volatility hydrocarbons such as ethylene as a bottoms product, but a high content of impurity gases such as CO remain. Low-pressure cryogenic separators can produce and separate ethylene at high purity but with a consequently lower recovery rate. In addition, impurities such as methane or nitrogen may also be present in a gas stream feed to an oxocarbon electrolyzer. These can act as inert gases in the system which eventually can build up in the combined system as CO or certain product gases are recycled back to the electrolyzer.

The process flow using an integrated system of an electrolyzer with multiple separators enables efficient production of high-purity ethylene while simultaneously providing routes to produce commercially viable high-pressure hydrogen while maximizing eventual conversion of CO (and CO₂) to ethylene. The use of a dual-pressure cryogenic separator reduces energy consumption compared to conventional cryogenic distillation schemes as the configuration allows for effective integration of refrigeration among the product and recycle stream, and the low pressure cryogenic stage eliminates the need for large reflux volumes or additional condensation or compression stages.

In specific embodiments of the invention, a dual-pressure cryogenic separation system is provided downstream of an oxocarbon electrolyzer. The system includes a high-pressure gas/liquid separator that separates the cooled product gas stream into a hydrogen-rich vapor stream and an ethylene-rich liquid stream. The ethylene-rich liquid stream is then expanded through a Joule-Thompson valve to produce a partially vaporized stream, which is fed into a low-pressure stripping column. The stripping column separates the stream into a purified ethylene bottom product and a CO-rich overhead stream. The hydrogen-rich vapor stream and the CO-rich overhead stream can both be recycled to the electrolyzer input to maximize carbon utilization. This configuration provides several benefits. First, it produces a purified ethylene stream at greater than 99.95% purity with high recovery yields suitable for downstream polymerization or oxidation processes. Second, it allows the hydrogen-rich stream to be recovered at a high pressure compatible with downstream hydrogen purification without additional compression. Third, it produces a CO-rich stream at a pressure and composition suitable for direct recycling to the electrolyzer. Finally, the integration of refrigeration between the various gas streams reduces the overall energy consumption per ton of ethylene compared to conventional cryogenic separation methods. In specific embodiments, the system can include one or more purge lines or other separators to remove inert impurities from recycled gases before they can build up to a high level.

In specific embodiments of the invention, a system is provided. The system includes an electrolyzer configured to receive an input feed stream comprising carbon monoxide and to produce a product gas stream comprising ethylene, carbon monoxide, and hydrogen. The system further includes a refrigeration system configured to cool the gas stream to condense the ethylene into a liquid, a gas/liquid separator configured to receive a gas stream at high pressure and to separate the cooled gas stream into a hydrogen-rich vapor stream and an ethylene-rich liquid stream, an expansion valve configured to reduce the pressure of the ethylene-rich liquid stream to produce a partially-vaporized stream, and a low-pressure stripping column configured to separate the partially-vaporized stream into a purified ethylene stream and a carbon monoxide-rich stream. The system further comprises at least one conduit configured to recycle at least one of a portion of the hydrogen-rich vapor stream and a portion of the carbon monoxide-rich stream to the input feed of the electrolyzer.

In specific embodiments of the invention, a cryogenic separation system is provided. The cryogenic separation system includes a refrigeration system configured to cool a gas stream comprising ethylene, carbon monoxide, and hydrogen to condense the ethylene into a liquid, a gas/liquid separator configured to receive a gas stream at high pressure and to separate the cooled gas stream into a hydrogen-rich vapor stream and an ethylene-rich liquid stream, an expansion valve configured to reduce the pressure of the ethylene-rich liquid stream to produce a partially-vaporized stream, and a low-pressure stripping column configured to receive the partially-vaporized stream at its top section and to separate it into a purified ethylene stream at the bottom and a carbon monoxide-rich stream at the top. The cryogenic separation system produces at least three distinct output streams, including (i) the hydrogen-rich vapor stream from the gas/liquid separator, (ii) the purified ethylene stream from the stripping column, and (iii) the carbon monoxide-rich stream from the stripping column.

In specific embodiments of the invention, a method is provided. The method includes refrigerating a gas stream comprising ethylene, carbon monoxide, and hydrogen to condense at least most of the ethylene into a liquid, separating the gas stream in a gas/liquid separator operating at high pressure into a hydrogen-rich vapor stream and an ethylene-rich liquid stream, and expanding the ethylene-rich liquid stream through an expansion valve to form a partially-vaporized stream. The method also comprises feeding the partially-vaporized stream at or near the top of a low-pressure stripping column, separating the partially-vaporized stream within the low-pressure stripping column to produce a purified ethylene stream and a carbon monoxide-rich stream, and recovering as outputs (i) the hydrogen-rich vapor stream, (ii) the purified ethylene stream, and (iii) the carbon monoxide-rich stream.

### BRIEF DESCRIPTION OF THE DRAWINGS

The accompanying drawings illustrate various embodiments of systems, methods, and various other aspects of the disclosure. A person with ordinary skills in the art will appreciate that the illustrated element boundaries (e.g., boxes, groups of boxes, or other shapes) in the figures represent one example of the boundaries. It may be that in some examples one element may be designed as multiple elements or that multiple elements may be designed as one element. In some examples, an element shown as an internal component of one element may be implemented as an external component in another, and vice versa. Furthermore, elements may not be drawn to scale. Non-limiting and non-exhaustive descriptions are described with reference to the following drawings. The components in the figures are not necessarily to scale, emphasis instead being placed upon illustrating principles.

FIG. 1 provides a diagram of a system including a CO electrolyzer and a cryogenic separation system according to specific embodiments of the invention.

FIG. 2 provides a diagram of a system including a CO/CO₂ electrolyzer and a cryogenic separation system according to specific embodiments of the invention.

FIG. 3 provides a diagram of a cryogenic separation system according to specific embodiments of the invention.

FIG. 4 provides a diagram containing several variants of CO/hydrogen separators according to various specific embodiments of the invention.

FIG. 5 provides a diagram of a process describing a method of cryogenic separation of a gas mixture.

### DETAILED DESCRIPTION

Reference will now be made in detail to implementations and embodiments of various aspects and variations of systems and methods described herein. Although several exemplary variations of the systems and methods are described herein, other variations of the systems and methods may include aspects of the systems and methods described herein combined in any suitable manner having combinations of all or some of the aspects described. Methods and systems related to CO electrolyzers and separators in accordance with the summary above are disclosed in detail herein. Electrolyzers and separators as used with the approaches discussed herein can be configured with various architectures and may also be integrated into larger systems.

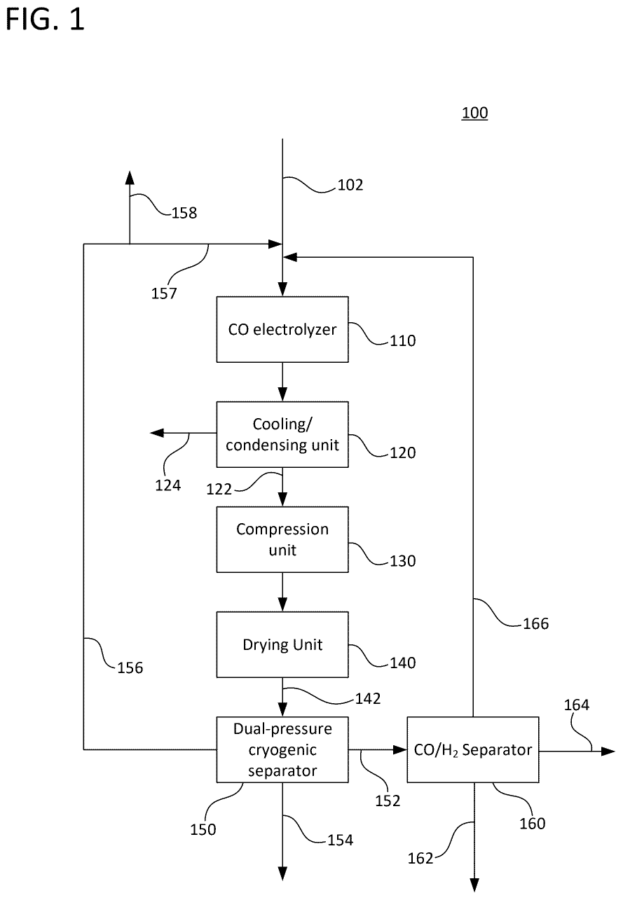

FIG. 1 illustrates a process flow and elements of a system 100 for converting CO to ethylene and separating product streams according to specific embodiments. The system 100 includes a CO electrolyzer 110 configured to receive an input gas stream 102 comprising CO and to convert at least a portion of the CO into ethylene. In specific embodiments, two main global reactions create ethylene and hydrogen according to equations 1 and 2 below.

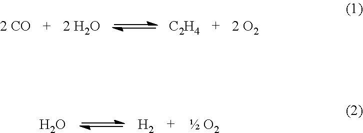

The product gas stream exiting the CO electrolyzer 110 comprises ethylene, unconverted CO, and hydrogen. In specific embodiments, the gas stream is 5-50 mol percent (mol %) CO, 20-70% mol % ethylene, and 20-70 mol % hydrogen, where mol % is a unit of concentration that expresses the fraction of a component's amount in moles relative to the total number of moles in a mixture, multiplied by 100. Optionally, it can comprise additional components depending on the source gas and reaction conditions in the electrolyzer, including, for example, water vapor, nitrogen, methane, hydrogen sulfide, or CO₂. The gas stream may be saturated with water vapor at low temperatures (e.g., <80° C.) and at low to medium pressure (e.g., <10 bar). If the product gas contains an acid gas such as hydrogen sulfide or CO₂, it can be removed optionally with an acid gas removal system (not shown).

The product gas stream from the CO electrolyzer 110 is directed to a cooling/condensing unit 120. The cooling/condensing unit 120 operates to condense water vapor and any higher-boiling components. One goal of this is to remove the majority of water and other constituents with a higher boiling point than ethylene. The condensed liquid phase is removed through output stream 124, while the cooled gas stream 122 is passed downstream to a compression unit 130, where the gas stream is to a high enough pressure (e.g. 30-80 bars) for cryogenic separation in later stages. The compressed gas is then passed to a drying unit 140, which removes residual water vapor and other condensable species. In specific embodiments, the drying unit 140 may comprise, but is not limited to, a temperature swing adsorption (TSA) unit, or other drying technologies effective to produce a substantially dry gas stream. The drying unit also can remove trace portions of hydrocarbons or other impurities with a higher boiling point than ethylene that would otherwise separate out in the bottoms ethylene product of the cryogenic separator downstream and potentially compromise its purity.

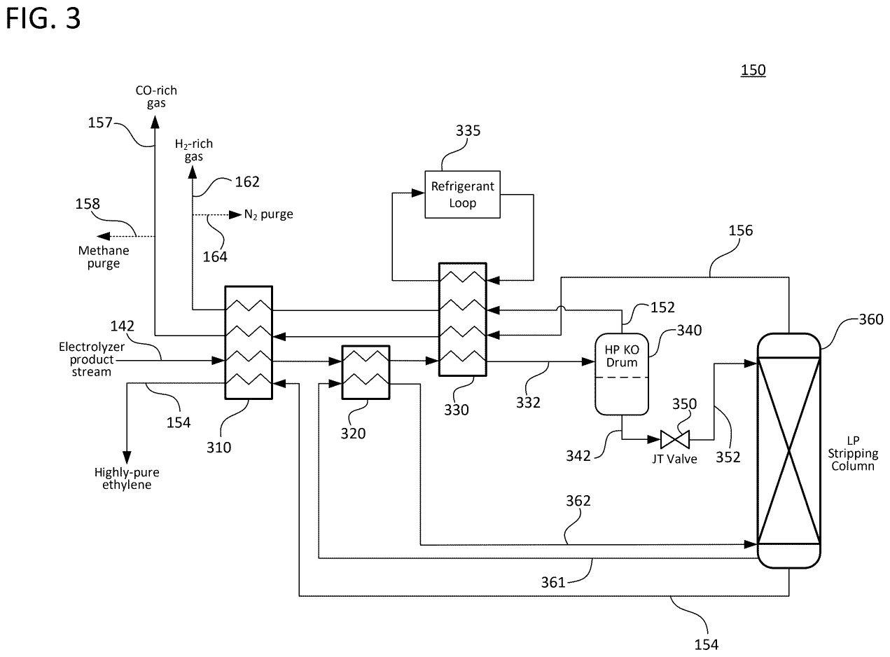

The dried and compressed gas stream 142 is then routed to a dual-pressure cryogenic separator 150. The dual-pressure cryogenic separator 150 will be described in more detail with respect to FIG. 3. In short, the separator 150 is configured to split the gas stream into at least a hydrogen-rich stream 152, a purified ethylene product stream 154, and a CO-rich stream 156. In specific embodiments, the dual-pressure cryogenic separator 150 includes a high-pressure knockout drum that separates the bulk of hydrogen from the ethylene and CO, an expansion valve that reduces the pressure of the ethylene-rich liquid stream, and a low-pressure stripping column that separates the expanded liquid stream into purified ethylene bottoms liquid and a CO-rich overhead.

The hydrogen-rich stream 152 from the dual-pressure cryogenic separator 150 is directed to a CO/hydrogen separator 160. The CO/hydrogen separator 160 may include one or more of, for example, a PSA unit, a membrane separation unit, or a cryogenic distillation unit configured to separate the hydrogen-rich vapor stream into a purified hydrogen stream 162, an inert gas stream such as purge nitrogen stream 164, and a CO stream 166. In certain embodiments, the purified hydrogen stream 162 may be valorized for downstream use, while the CO stream 166 may be recycled directly or indirectly to the input gas stream 102 of the CO electrolyzer 110. In specific embodiments, the CO/hydrogen separator 160 can be wholly or partially bypassed, where at least part of the hydrogen-rich stream is directly recycled to the input gas stream 102 of the CO electrolyzer 110.

The CO-rich stream 156 exiting the dual-pressure cryogenic separator 150 can be recycled upstream to the CO electrolyzer 110. Recycling this stream provides significant benefits. The CO-rich stream 156 still contains unconverted CO along with some fraction of hydrogen and ethylene. By returning both CO-rich stream 156 and CO stream 166 to the electrolyzer 110, the overall CO conversion efficiency of the system is increased while minimizing the loss of ethylene contained in the recycle stream as it will eventually be separated into a product output.

In some embodiments, a purge stream 158 may be withdrawn from the CO-rich stream 156 upstream of the electrolyzer 110, with the remainder 157 returned to the input of the electrolyzer. This purge stream 158 provides a mechanism for removing undesired buildup of other inert gases (e.g. methane) that may otherwise accumulate in the electrolyzer and that tend to separate with the CO-rich stream 156. In specific embodiments, the purge stream 158 may be treated in an auxiliary purification process or used as a low-value fuel gas. In specific embodiments, operating temperatures in the separator 150 may send a significant portion of the methane present (e.g. 10-50 mol %) into the hydrogen-rich stream 152. In this case, methane can be purged along with nitrogen or otherwise separated out in the CO/hydrogen separator 160. This may also be sufficient to prevent methane from building up in the system overall.

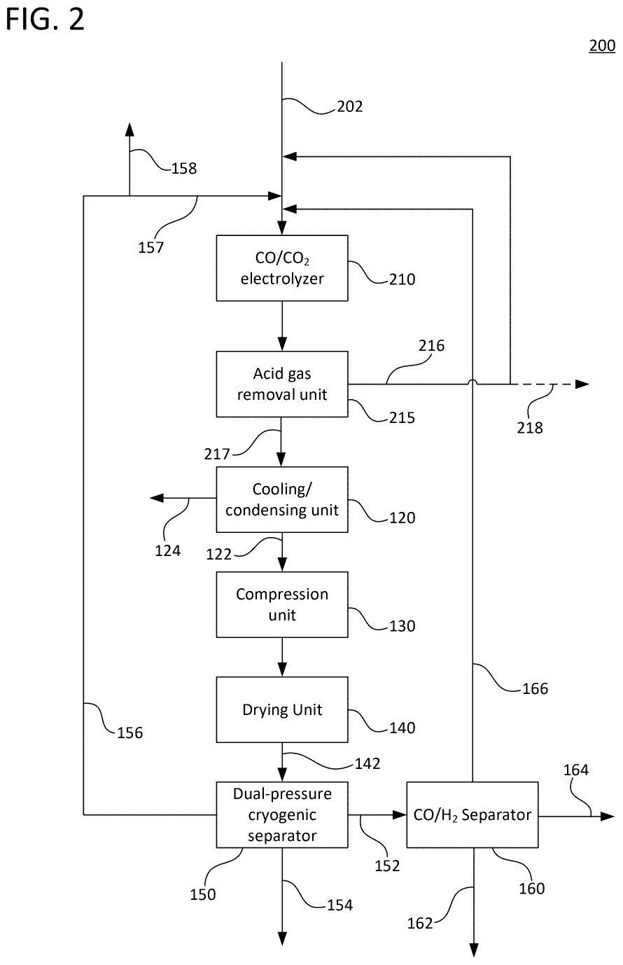

FIG. 2 illustrates a process flow and elements of a system 200 for converting CO and CO₂ to ethylene and separating product streams according to specific embodiments. The system 200 includes a CO/CO₂ electrolyzer 210 configured to receive an input gas stream 202 comprising CO₂ and to convert at least a portion of the CO₂ into ethylene. In specific embodiments, input gas stream 202 can also optionally comprise CO as well, where the electrolyzer 210 can be configured to convert at least a portion of the CO to ethylene. In specific embodiments, another global reaction creates ethylene according to equation 3 below in addition ethylene and hydrogen produced according to equations 1 and 2.

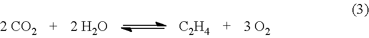

Whether the input gas stream 202 contains CO or not, the product gas stream exiting the CO/CO₂ electrolyzer 210 comprises ethylene, unconverted CO₂, CO, and hydrogen. In specific embodiments, CO in the product gas stream can be unconverted CO from the input gas stream 202. CO in the product gas stream can also be formed as a byproduct conversion from CO₂ in the electrolyzer 210 as a competing side reaction. Even if the catalyst and other features are tuned to maximize the conversion of CO₂ to ethylene, some CO is likely produced. The product gas stream is routed to an acid gas removal unit 215. CO2 and any hydrogen sulfide that might be present are separated into a stream 216, where ethylene, hydrogen, CO, and other components are passed along to the cooling/condensing unit 120 in stream 217. The CO2 stream 216 can be optionally discarded or otherwise processed to an output stream 218, but is otherwise recycled back to the input of the electrolyzer 210. In specific embodiments, the acid gas removal unit 215 can be placed after the cooling/condensing unit 120.

The remaining components and process as shown in FIG. 2 are substantially the same as that already described with respect to FIG. 1 and system 100 starting with the cooling/condensing unit 120 and including the cryogenic separator 150. Notably, a CO-rich recycle stream 156 and the CO portion of recycle stream 166 can be recycled to the input of the electrolyzer 210. In this manner, the CO/CO₂ electrolyzer 210 can be used to convert recycled CO to ethylene even if it was otherwise optimized to convert CO₂ to ethylene as well.

FIG. 3 illustrates a dual-pressure cryogenic separator 150 according to specific embodiments. The cryogenic separator 150 is integrated downstream of the CO electrolyzer product stream 142 and is configured to achieve efficient separation of ethylene from CO and hydrogen. The separation is carried out in multiple stages of cooling, phase separation, and cryogenic stripping. The electrolyzer product stream 142, after upstream drying and compression, is first directed into a sequence of heat exchangers 310, 320, and 330. The heat exchangers can be made of brazed aluminum, but could also be constructed of copper, nickel, stainless steel, or other metals with reasonable thermal conductivity. Within the first heat exchanger 310, the product stream 142 is precooled using available cold energy from ethylene product stream 154, hydrogen-rich stream 152, and CO-rich stream 156, achieving substantial temperature reduction while minimizing external refrigeration requirements. The temperature can be cooled down to no more than −65° C., for example, at this stage based on the available cooling capacities of the output streams. The precooled stream 142 is then further chilled in a second heat exchanger 320, where heat integration with the bottoms product (ethylene product stream 154) from the low-pressure stripping column 360 allows the heat exchanger 320 to operate as a reboiler for the column. The expected reboiler temperature can be around −70° C. depending on the CO/ethylene ratio at the top of the stripping column 360, so the exit temperature may be down to −65° C. considering an approach temperature of 3-5° C. in the heat exchanger 320. The third heat exchanger 330 supplied with additional cold from a refrigerant loop 335 along with hydrogen-rich stream 152 and CO-rich stream 156, lowers the temperature of the stream to cryogenic levels (e.g., −90° C. to −130° C.), producing a partially-condensed stream 332. The exact temperature can be selected depending on the input mix composition as well as separation objectives. For instance, though ethylene purity may be of primary concern, in some circumstances this temperature can be chosen to also maximize hydrogen purity. In specific embodiments, the refrigerant loop system can be based on, but not limited to, a cascade refrigeration system using several hydrocarbon refrigerants (propane, ethylene, methane), a single mixed refrigerant loop, a dual mixed refrigerant loop, or a nitrogen or argon refrigerant loop.

The partially-condensed stream 332 exiting the third heat exchanger 330 is introduced into a gas/liquid separator, in this example a high-pressure knockout drum (HP KO) 340, but this could also be a cold box phase separator. The HP KO drum 340, operating at pressures ranging, for example, from about 30 to 80 bar, separates the stream into a hydrogen-rich stream 152 overhead and an ethylene-rich liquid stream 342, which can comprise ethylene and CO. The overhead hydrogen-rich stream 152 passes back through heat exchangers as previously described, and hydrogen stream 162 may be directed to a downstream CO/hydrogen separator 160. Although a purge nitrogen stream 164 can be separated out using various techniques in the CO/hydrogen separator 160, in specific embodiments, a purge nitrogen stream 164 can be split directly before the CO/hydrogen separator 160 which purges a small amount of gas sufficient to prevent any gases such as nitrogen present in the input gas stream 102 that would otherwise separate with the hydrogen from accumulating in the loop. While a portion of the CO in the electrolyzer product gas passes into the liquid stream 342, a large portion remains in gaseous form and returns with the hydrogen-rich stream 152. In specific embodiments, the purge stream 164 can be configured to purge hydrogen from the system. A purge stream for hydrogen can allow the system to recycle various product and unreacted gases without allowing hydrogen to build up in the system. This could also allow the CO/hydrogen separator 160 to be partially or fully bypassed as mentioned with respect to FIG. 1. This can also be effective in situations where the electrolyzer has been configured to produce less hydrogen relative to ethylene.

The liquid stream 342 from the HP KO drum 340 are depressurized through a Joule-Thompson (JT) valve 350, reducing the pressure to, for example, about 1 to 10 bar. This expansion provides additional refrigeration to bring the temperature to a desired range for separation in a stripping column 360. This provides additional liquid that can be used as internal reflux within the downstream low-pressure stripping column 360 while partially vaporizing the mixture. The two-phase mixture 352 formed after expansion enters the low pressure (LP) stripping column 360 from the top, where the vapor fraction rises and exits as a CO-rich stream 156, while the liquid fraction descends and undergoes fractionation. The top of the LP stripping column 360 can be operated from between −100° C. to −140° C., and at the ethylene boiling point at the bottom of the column. The LP column 360 thus separates a highly pure ethylene product stream 154 from the bottom, which can exceed 99.95% purity, while the overhead CO-rich stream 156 can be heated through heat exchangers to ultimately be recycled upstream to the electrolyzer. A portion of the ethylene bottoms liquid 361 is sent through second heat exchanger 320 and returned as stream 362 back to the bottom of the column as a reboiler. Ethylene product stream 154 can be directly used in a variety of downstream synthesis processes including but not limited to production of ethylene oxide, polymers (e.g. high-density polyethylene, ethylene-vinyl acetate), or monomers (e.g. vinyl acetate, vinyl chloride). Many of these applications require a high-purity input. Ethylene specifications for CO content in the most stringent cases cannot exceed 1-2 parts per million (ppm), but in general the specification rarely allows more than 10 ppm CO. As used herein ppm refers to a dimensionless unit that expresses the concentration of one substance in a mixture as the number of parts of that substance per one million parts of the total. In specific embodiments, ethylene product stream 154 can be pumped at high pressure before being used to heat the inlet gas stream 142 in the first heat exchanger 310.

The integrated design of the cryogenic separation unit enables high ethylene recovery rates while simultaneously generating a high-pressure hydrogen-rich stream and a low-pressure CO-rich recycle stream. The dual pressure zones aid in the efficiency of the stripping column. As mentioned previously, a stripping column run at high pressure improves overall recovery of the bottoms liquid product by forcing more liquid to condense. This can have the undesired effect of bringing some of the top fraction into the bottoms liquid, in this case leading to an ethylene product with a significant amount of CO contamination. High pressure is limited by the critical pressure of ethylene in any case. A low-pressure column can be more effective in producing a high-purity bottoms liquid but then requires a high reflux rate to ensure product purity along with a reasonable recovery rate. The direct output of the CO electrolyzer product stream 142 into a cryogenic column would make this difficult to achieve, because the ratio of the CO/hydrogen content results in a low dewpoint. An additional condenser along with temperatures needed to produce high reflux would make this highly energy intensive. By first separating most of the hydrogen from the liquid stream 342 before lowering the pressure, this problem is greatly reduced. More of the incoming CO and residual hydrogen stay at the top of the column, while the liquid ethylene entering provides sufficient reflux for purification without the need for a condenser at the top of the column.

Unlike many commonly used cryogenic separators, in specific embodiments the stream after the JT valve is not added in the middle of the column for later stripping and rectification but can act mostly as a stripping column by adding the ethylene-rich liquid at the top of the column. This provides more time for reflux and purification of the ethylene product. One specification of the separator 150 is to produce a highly pure ethylene product that can be directly used as a feedstock for other processes. An additional specification is for high recovery of the ethylene that enters the system (e.g., >90% recovery). However, a small amount of ethylene that exits with the CO-rich stream 156 is acceptable. This can have synergistic effects depending on the design of the CO-electrolyzer. This will be discussed later with regard to other separators, but in specific embodiments, additional primary and secondary products recycled to an electrolyzer input stream along with unreacted CO may result in improved performance of the electrolyzer.

Table 1 below shows the material balance through the cryogenic separator 150 in one particular example. This example is not meant to be limiting but illustrates the possible flow of components at a particular set of temperatures, pressures, and flow rates through the system, along with an exemplary CO electrolyzer output gas stream run under specific conditions. The row labeled FIG. 3 Location refers to the figure numbering in FIG. 3 of the stream at a particular element.

TABLE 1
Material balance in the cryogenic separator

| | | | | | | | |
|---|---|---|---|---|---|---|---|
| FIG. 3 Location | 142 | 232 | 152 | 242 | 252 | 156 | 154 |
| Vapor fraction | 1 | 0.62 | 1 | 0 | 0.10 | 1 | 0 |
| Temperature (° C.) | 35 | −117 | −117 | −117 | −120 | −117 | −74 |
| Pressure (bar_g) | 44.0 | 43.7 | 43.7 | 43.7 | 3.5 | 3.5 | 3.5 |
| Molar flow (kmol/h) | 527 | 527 | 328 | 199 | 199 | 29 | 170 |
| Methane (mol %) | 1.0 | 1.0 | 0.8 | 1.4 | 1.4 | 9.7 | 0.0 |
| Hydrogen (mol %) | 43.1 | 43.1 | 68.0 | 2.1 | 2.1 | 14.4 | 0.0 |
| CO (mol %) | 20.7 | 20.7 | 27.7 | 9.1 | 9.1 | 63.2 | 0.0 |
| Ethylene (mol %) | 34.1 | 34.1 | 2.0 | 87.1 | 87.1 | 10.8 | 100.0 |
| Nitrogen (mol %) | 1.1 | 1.1 | 1.6 | 0.3 | 0.3 | 2.0 | 0.0 |

In this example, an ethylene purity of nearly 100% is achievable with an ethylene recovery rate through the separator of >94%. This example also shows that under these conditions, some constituents appear in several streams. For instance, though the primary component of CO-rich stream 156 is CO, a much greater amount of CO is present in the hydrogen-rich stream 152. Recycling of both streams results in the highest recovery rate of CO for eventual conversion to ethylene. Impurity streams of methane and nitrogen can be present in hydrogen-rich stream 152 and CO-rich stream 156, depending on separation conditions, so separation and removal of these streams (and where this should take place in the system) should be adjusted based on measured amounts in each stream.

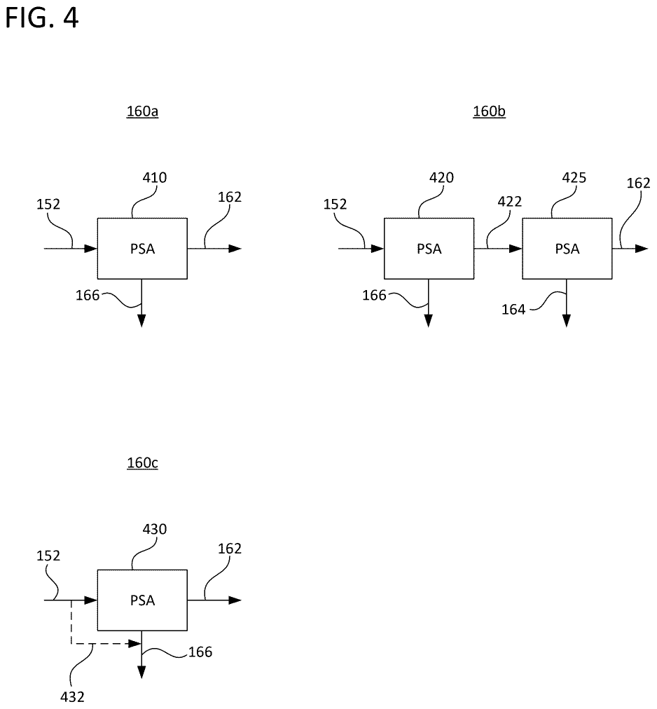

As previously mentioned with respect to embodiments found in FIG. 1, various options are available for CO/hydrogen separator 160. FIG. 4 illustrates several of these options according to specific embodiments. View 160a shows a single stage separator, which is shown here as a PSA 410, but could be a membrane separator. A single stage separator may be used in situations where a nitrogen purge is done separately from the CO/hydrogen separator 160. In one example, PSA 410 can be tuned to provide a reasonably pure hydrogen stream 162 as a permeate, while having a retentate CO stream 166 that comprises CO (and any ethylene present) along with some hydrogen. If higher purity hydrogen is desired as an output stream, additional purification stages can be added (not shown). Single stage separators can be configured so that either permeate or retentate have a higher purity level. For example, the PSA 410 could be run so that only hydrogen passed through but the flow is turned off well before breakthrough of CO or other components. Following this, the PSA is purged with additional hydrogen to release the retentate and remove any CO from void spaces. CO stream 166 may be high in CO, but also can contain an appreciable amount of hydrogen as well. This is not necessarily a drawback when the separators are integrated fully into a system containing a CO electrolyzer 110. CO electrolyzers configured to produce ethylene as a primary product can also produce hydrogen as a desirable secondary product. Recycling unreacted CO to the input gas stream 102 drives up the efficiency of conversion to ethylene and also increases the gas flow through the electrolyzer. However, electrolyzer catalysts can become flooded or oversaturated with CO, and so one or more relatively inert gases such as the product gases ethylene and hydrogen can set the mol % of incoming CO relative to CO in the input gas stream 102 to a desirable level for optimum conversion efficiency.

View 160b of FIG. 4 shows a dual-stage separator including a purge stage according to another embodiment. In this example, the retentate of PSA 420 comprises CO, and some ethylene and hydrogen. Permeate 422 passes relatively pure hydrogen along with any nitrogen present. PSA 425 is tuned to retain nitrogen and pass hydrogen. Purge nitrogen stream 164 contains most of the nitrogen separated by the cryogenic separator 150 and can be purged with minimal loss of unreacted CO or hydrogen. In specific embodiments, PSA 420 can be configured to also pass methane while PSA 425 is configured to retain methane. This allows CO/hydrogen separator 160 in the configuration shown in view 160b to act as a purge separator for both nitrogen and methane in the system, possibly removing the need for a separate methane purge stream 158.

View 160c of FIG. 4 shows a similar separator as view 160a but includes an additional bypass line 432 that bypasses PSA 430. As previously mentioned, some or all of the hydrogen-rich stream 152 can bypass a CO/hydrogen separator. The amount bypassed can be controlled to adjust the ratio of CO/hydrogen entering the CO electrolyzer. If all of the hydrogen-rich stream 152bypasses the CO/hydrogen separator then the hydrogen can be purged along with the nitrogen or methane.

As depicted in FIG. 1, the hydrogen-rich stream 152 enters the CO/hydrogen separator 160. In specific embodiments, hydrogen-rich stream 152 and CO-rich streams can be combined before entering the CO/hydrogen separator 160. This could be implemented with any of the separators shown in views 160a, 160b, or 160c. Because the PSAs can be configured to adsorb or otherwise retain ethylene in addition to CO, most or all of the CO and ethylene can be recycled through the electrolyzer for eventual conversion or valorization.

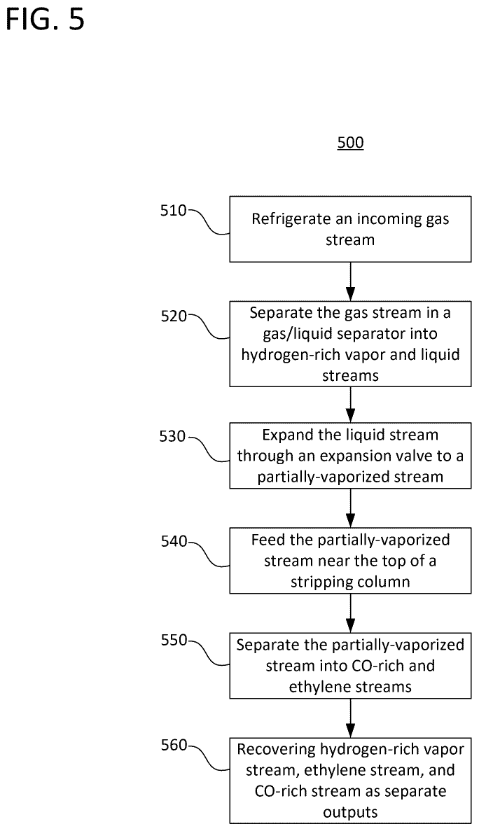

FIG. 5 describes a process 500 for separating a gas stream comprising ethylene, hydrogen, and CO according to specific embodiments. In step 510, the incoming gas stream is first refrigerated so that most of the ethylene in the stream is condensed to a liquid state. In step 520, the refrigerated stream is separated in a gas/liquid separator (such as a knockout drum) so that the hydrogen-rich stream exits the top, and an ethylene-rich liquid exits the bottom. In step 530, the liquid stream is expanded through an expansion valve. This lowers the pressure of the stream and slightly cools it further. The expansion of the stream partially-vaporizes a portion of the stream. In step 540, the partially-vaporized stream is fed at or near the top of a stripping column. In step 550, the stripping column separates the partially-vaporized stream into two outputs, a CO-rich gas stream out the top, and a purified ethylene stream from the bottom. In step 560, the hydrogen-rich vapor stream, the ethylene stream, and the CO-rich stream are recovered as separate outputs.

Different systems and methods for gas separation and integration into electrolyzer systems are described in detail in this disclosure. The methods and systems disclosed in this section are nonlimiting embodiments of the invention, are provided for explanatory purposes only, and should not be used to constrict the full scope of the invention. It is to be understood that the disclosed embodiments may or may not overlap with each other. Thus, part of one embodiment, or specific embodiments thereof, may or may not fall within the ambit of another, or specific embodiments thereof, and vice versa. Different embodiments from different aspects may be combined or practiced separately. Many different combinations and sub-combinations of the representative embodiments shown within the broad framework of this invention, that may be apparent to those skilled in the art but not explicitly shown or described, should not be construed as precluded.

While the specification has been described in detail with respect to specific embodiments of the invention, it will be appreciated that those skilled in the art, upon attaining an understanding of the foregoing, may readily conceive of alterations to, variations of, and equivalents to these embodiments. These and other modifications and variations to the present invention may be practiced by those skilled in the art, without departing from the scope of the present invention, which is more particularly set forth in the appended claims.

What is claimed is:

1. A system for converting carbon monoxide to ethylene comprising:
an electrolyzer configured to receive an input feed stream comprising carbon monoxide and to produce a product gas stream comprising ethylene, carbon monoxide, and hydrogen;
a refrigeration system configured to cool the product gas stream with a temperature sufficiently low enough to condense the ethylene into a liquid;
a gas/liquid separator configured to receive the product gas stream at a high pressure, and to separate the product gas stream into a hydrogen-rich vapor stream and an ethylene-rich liquid stream, the ethylene-rich liquid stream including the liquid;
an expansion valve fluidly coupled to a liquid stream output on the gas/liquid separator and configured to reduce the pressure of the ethylene-rich liquid stream to produce a partially-vaporized stream;
a low-pressure stripping column that: (i) is fluidly coupled to the expansion valve; (ii) receives the partially-vaporized stream at its top section; and (iii) is operable to separate the partially-vaporized stream into a purified ethylene stream, recovered as a bottom product of the low-pressure stripping column, and a carbon monoxide-rich stream, recovered as an overhead product of the low-pressure stripping column; and
at least one conduit configured to recycle at least one of a portion of the hydrogen-rich vapor stream or a portion of the carbon monoxide-rich stream to the input feed stream of the electrolyzer.

2. The system of claim 1, further comprising:
one or more condensing and drying units to remove substantially all constituents with a higher boiling point than ethylene from the product gas stream.

3. A cryogenic separation system for processing a gas stream comprising ethylene, carbon monoxide, and hydrogen, the cryogenic separation system comprising:
a refrigeration system configured to cool the gas stream with a temperature sufficiently low enough to condense the ethylene into a liquid;
a gas/liquid separator configured to receive the gas stream at a high pressure, and to separate the gas stream into a hydrogen-rich vapor stream and an ethylene-rich liquid stream, the ethylene-rich liquid stream including the liquid;
an expansion valve fluidly coupled to a liquid stream output on the gas/liquid separator and configured to reduce the pressure of the ethylene-rich liquid stream to produce a partially-vaporized stream; and
a low-pressure stripping column fluidly coupled to the expansion valve that receives the partially-vaporized stream at its top section and operable to separate the partially-vaporized stream into a purified ethylene stream, recovered as a bottom product of the low-pressure stripping column, and a carbon monoxide-rich stream, recovered as an overhead product of the low-pressure stripping column;
wherein the cryogenic separation system produces at least three distinct output streams: (i) the hydrogen-rich vapor stream from the gas/liquid separator, (ii) the purified ethylene stream from the low-pressure stripping column, and (iii) the carbon monoxide-rich stream from the low-pressure stripping column.

4. The cryogenic separation system of claim 3, wherein the gas/liquid separator is a knockout drum or a cold box phase separator.

5. The cryogenic separation system of claim 3, further comprising:
a carbon monoxide electrolyzer configured to receive a feed comprising carbon monoxide and to produce a product gas stream comprising ethylene, carbon monoxide, and hydrogen, wherein the product gas stream is directed from the carbon monoxide electrolyzer to be the gas stream inputted to the cryogenic separation system.

6. The cryogenic separation system of claim 5, wherein the carbon monoxide electrolyzer further produces water vapor in the gas stream, and the cryogenic separation system further comprises a condenser and a drying unit upstream of the cryogenic separation system for removing water from the product gas stream.

7. The cryogenic separation system of claim 5, wherein the carbon monoxide electrolyzer further produces one or more acid gases comprising at least one of carbon dioxide and hydrogen sulfide in the product gas stream, and the cryogenic separation system further comprises an acid gas removal unit upstream of the cryogenic separation system for removing the one or more acid gases from the product gas stream.

8. The cryogenic separation system of claim 5, wherein the product gas stream of the carbon monoxide electrolyzer comprises from about 5 mol % to about 50 mol % carbon monoxide, from about 20 mol % to about 70 mol % ethylene, and from about 20 mol % to about 70 mol % hydrogen.

9. The cryogenic separation system of claim 5, wherein the carbon monoxide-rich stream exiting the low-pressure stripping column is recycled to the carbon monoxide electrolyzer as a feed stream.

10. The cryogenic separation system of claim 9, wherein the carbon monoxide-rich stream exiting the low-pressure stripping column further comprises methane.

11. The cryogenic separation system of claim 10, further comprising a purge line configured to at least periodically remove methane from the carbon monoxide-rich stream recycled from the low-pressure stripping column.

12. The cryogenic separation system of claim 5, wherein the carbon monoxide-rich stream recycled to the carbon monoxide electrolyzer comprises up to about 50 mol % ethylene.

13. The cryogenic separation system of claim 5, wherein recycling the carbon monoxide-rich stream increases overall carbon monoxide conversion efficiency of the carbon monoxide electrolyzer.

14. The cryogenic separation system of claim 5, further comprising a purge line configured to at least periodically remove nitrogen from the hydrogen-rich vapor stream.

15. The cryogenic separation system of claim 5, further comprising a hydrogen/carbon monoxide separation unit configured to receive the hydrogen-rich vapor stream from the gas/liquid separator and to separate the hydrogen-rich vapor stream into a purified hydrogen stream and a carbon monoxide stream.

16. The cryogenic separation system of claim 15, wherein:
the hydrogen/carbon monoxide separation unit is configured to also separate at least one impurity stream from the hydrogen-rich vapor stream; and
the at least one impurity stream comprises at least one of nitrogen and methane.

17. The cryogenic separation system of claim 15, wherein the hydrogen/carbon monoxide separation unit includes one or more elements selected from the group consisting of: a pressure swing adsorption (PSA) unit, a cryogenic distillation unit, and a membrane separation unit.

18. The cryogenic separation system of claim 15, wherein a carbon monoxide stream separated from the hydrogen-rich vapor stream is recycled as an input feed to the carbon monoxide electrolyzer.

19. The cryogenic separation system of claim 5, wherein the hydrogen-rich vapor stream from the gas/liquid separator is directly recycled to the carbon monoxide electrolyzer as an input feed.

20. The cryogenic separation system of claim 19, wherein the hydrogen-rich vapor stream recycled to the carbon monoxide electrolyzer comprises both hydrogen and carbon monoxide.

21. The cryogenic separation system of claim 20, wherein recycling the hydrogen-rich vapor stream increases the overall conversion efficiency of carbon monoxide in the carbon monoxide electrolyzer.

22. The cryogenic separation system of claim 5, wherein:
the carbon monoxide electrolyzer is also configured to receive carbon dioxide as a feed gas; and
the product gas stream further comprises unreacted carbon dioxide.

23. The cryogenic separation system of claim 22, wherein:
the carbon monoxide electrolyzer is configured to convert at least a portion of both carbon monoxide and carbon dioxide in the feed gas into ethylene.

24. The cryogenic separation system of claim 3, wherein the refrigeration system comprises at least one heat exchanger configured to cool the gas stream using refrigeration from at least one of the purified ethylene stream, the hydrogen-rich vapor stream, and the carbon monoxide-rich stream.

25. The cryogenic separation system of claim 24, wherein at least one of the at least one heat exchanger is configured to act as a reboiler for the low-pressure stripping column.

26. A method of separating a gas stream comprising ethylene, carbon monoxide, and hydrogen, the method comprising:
refrigerating the gas stream with a temperature sufficiently low enough to condense at least a majority of the ethylene into a liquid state;
separating the gas stream in a gas/liquid separator operating at a high pressure into a hydrogen-rich vapor stream and an ethylene-rich liquid stream;
expanding the ethylene-rich liquid stream through an expansion valve to reduce the pressure of the ethylene-rich liquid stream to form a partially-vaporized stream;
feeding the partially-vaporized stream at or near the top of a low-pressure stripping column;
separating the partially-vaporized stream within the low-pressure stripping column to produce a purified ethylene stream as a bottom product and a carbon monoxide-rich stream as an overhead product; and
recovering as outputs: (i) the hydrogen-rich vapor stream; (ii) the purified ethylene stream; and (iii) the carbon monoxide-rich stream.
